(19) United States
(12) Patent Application Publication
(10) Pub. No.: US 2023/0295612 A1
(43) Pub. Date: Sep. 21, 2023

(54) METHOD FOR SCREENING FOR BIOACTIVE NATURAL PRODUCTS

(71) Applicant: UNIVERSITY OF WARWICK, Coventry (GB)

(72) Inventors: Gregory L. CHALLIS, Coventry (GB); Douglas ROBERTS, Coventry (GB)

(21) Appl. No.: 18/018,690

(22) PCT Filed: Jul. 30, 2021
(86) PCT No.: PCT/GB2021/051975

§ 371 Date: Jan. 30, 2023

(30) Foreign Application Priority Data
Jul. 31, 2020 (GB) 2011922.8

(51) Int. Cl. C12N 15/10; C12Q 1/689

(52) U.S. Cl. CPC C12N 15/1086; C12Q 1/689; C12N 15/1089; C12Q 2600/136

(57) ABSTRACT

The present invention relates to methods for screening for the presence of a biosynthetic gene cluster (BGC) in a cell, via the identification of proximal positive 5 regulatory genes, e.g. large ATP-binding regulators of the LuxR family (LAL) genes.

The present invention relates to methods for screening for the presence of a biosynthetic gene cluster (BGC) in a cell, via the identification of proximal positive regulatory genes, e.g. large ATP-binding regulators of the LuxR family (LAL) genes.

Many of the most commercially-successful chemical compounds in the pharmaceutical and agrochemical industries were discovered in microbes (e.g. penicillin and vancomycin); these are referred to as natural products. The traditional method for identifying leads for the development of new drugs and agrochemicals involves screening plant and microbial extracts for novel metabolites with a particular bioactivity. More recently, these latter methods have largely been supplanted by alternative techniques, such as high-throughput screening (HTS) of synthetic compound libraries and fragment-based design, both of which have been successful in identifying novel products.

There is still vast potential, however, for the discovery of novel natural products from plants and microorganisms.

Following the sequencing of a number of microbial genomes in the early 2000s, it was discovered that many Actinobacteria and filamentous fungi appeared to be capable of producing a far higher number of complex metabolites than was previously known. For example, following the sequencing of the soil bacterium Streptomyces coelicolor A3(2), an additional 16 previously-unidentified gene clusters were discovered, encoding enzymes such as non-ribosomal peptide synthetases (NRPSs), polyketide synthases (PKSs), terpene synthases and NRPS-independent siderophore synthetases. (Rutledge & Challis, 2015, Box 2). The metabolic products of these additional gene clusters were previously unknown, but bioinformatics-based predictions suggested that several were likely to encode products with novel structures.

Recent work using advanced genome sequencing techniques on soil microbes has shown that most of the natural products that microbes are capable of producing are not actually observed in laboratory cultures. This is because the genes which code for their production are “switched off” or are poorly expressed under standard laboratory conditions.

Numerous approaches have been used to try to activate these silent biosynthetic gene clusters (see, for example, Rutledge & Challis, (2015), Table 1). These approaches fall within two main classes: pleiotropic methods and pathway-specific methods. Pleiotropic methods include varying the growth conditions, engineering the transcription and translation machinery, manipulating global regulators and epigenetic changes. Pathway-specific methods include manipulating pathway-specific regulators, reporter-guided mutant selection, refactoring and heterologous expression.

The starting point for the above methods, however, is putatively to identify a cryptic gene cluster, generally by using a bioinformatics-based method based on screening for a gene cluster. One significant limitation of the above methods, therefore, is that they select in advance for the nature of the gene cluster, i.e. by only searching for sequences that have homology to previously-known gene clusters. Hence, by definition, such methods will not be capable of identifying new gene clusters which have low levels of sequence identify to known gene clusters.

There remains a need, therefore, for new screening methods that are capable of identifying new gene clusters which might have low levels of sequence identify or no sequence identity to known gene clusters. Such clusters might be capable of producing novel natural products which could form the basis for the development of novel medicaments, herbicides, insecticides and fungicides, inter alia.

Currently, no generalizable approaches exist for activating silent biosynthetic gene clusters (BGCs) in bacteria, although there are many isolated examples of BGCs being activated through culturing and engineering.

The inventors therefore systematically evaluated numerous methods for rational activation of silent BGCs identified in Actinobacterial genomes in an attempt to determine whether one approach could be turned into a generalizable method. Their efforts were, however, largely unsuccessful. A new approach was therefore required.

Many cryptic bacterial gene clusters have been found to be associated with transcriptional activators which induce expression of the gene cluster under appropriate environmental conditions. The inventors had the insight to consider whether—instead of searching for cryptic bacterial gene clusters—novel clusters could be found by searching for transcriptional activators. During the course of their investigations, the inventors noted that some cryptic bacterial gene clusters were associated with a transcriptional activator belonging to the LAL family (large ATP-binding regulators of the LuxR family). Initial investigations of this activator as a potential marker for cryptic bacterial gene clusters were not promising, for the following reasons, inter alia:
(i) Low levels of sequence homology between the known LAL genes meant that it was difficult to use standard bioinformatics approaches to screen sequence databases for new LAL genes. An initial search within an in-house set of 44 sequenced Actinobacterial genomes identified less than 20 hits.
(ii) The large size of the LAL genes (>3 kb) meant that they were difficult to manipulate and clone.
(iii) The high G+C content (˜70%) of the Actinobacterial LAL genes meant that they were difficult to synthesise and clone.

Hence, LAL genes did not look to be good candidates as a potential marker for cryptic bacterial gene clusters.

However, the inventors subsequently developed a novel bioinformatics screening approach using a Hidden Markov model, which was used to rescreen their databases, identifying over 100 potential gene clusters.

The inventors have also now demonstrated that it is possible to reduce the G+C content of the LAL-encoding genes by synthesising codon-altered versions of them, thus enabling the cloning and expression of the LAL activator. The use of low G+C content genes also had the effect of increasing transformation efficiency in some bacteria. Hence the inventors have now established that, contrary to their early expectations, LAL genes and other positive regulatory genes can indeed be used as a potential marker and activation tool for new cryptic bacterial gene clusters.

It is an object of the invention, therefore, to provide a method for screening for the presence of biosynthetic gene clusters in a bacterial cell. It is also an object of the invention to provide a method for the screening for the presence of a chemical entity in a bacterial cell.

In one embodiment, the invention provides a method for screening for the presence of a chemical entity in a bacterial cell, the method comprising the steps of:
(a) expressing a positive regulatory gene in a bacterial cell; and
(b) determining the presence of one or more chemical entities, other than the polypeptide which is encoded by the positive regulatory gene, whose expression level is increased in the bacterial cell after the expression of the positive regulatory gene, and optionally,
(c) isolating and/or identifying the chemical entity.

In a further embodiment, the invention provides a method for screening for the presence of a biosynthetic gene cluster in a bacterial cell, the method comprising the steps of:
(a) identifying the location of a nucleotide sequence coding for a positive regulatory gene within the nucleotide sequence of the genome of a bacterial cell; and
(b) analysing the nucleotide sequence of the cell genome in the proximity of the location of the nucleotide sequence of the identified positive regulatory gene in order to determine the presence of a nucleotide sequence which codes for a biosynthetic gene cluster.

Preferably, Steps (a) and/or (b) are implemented using a computer. Preferably, Step (a) is carried out using a Hidden Markov model.

In some embodiments, the method additionally comprises the step of:
(c) proposing a molecular structure for a product resulting from the expression of the biosynthetic gene cluster.
   Preferably, the above Step (c) is implemented using a computer.

In some embodiments, the method additionally comprises the steps of:
(d) obtaining a nucleic acid molecule whose nucleotide sequence comprises the nucleotide sequence of the biosynthetic gene cluster;
(e) expressing the nucleic acid molecule in a heterologous host cell;
(f) expressing the positive regulatory gene, or a derivative thereof, in the heterologous host cell;
   and optionally
(g) isolating and/or identifying a product resulting from the expression of the biosynthetic gene cluster.
   Steps (e) and (f) may be carried out in either order or simultaneously.

In some embodiments of the invention, the method for screening for the presence of a biosynthetic gene cluster in a bacterial cell is followed by Steps (a) and (b) of the method for screening for the presence of a chemical entity in a bacterial cell, wherein the methods refer to common bacterial gene clusters and common positive regulatory genes.

Preferably, the bacterial cells or heterologous host cells are Gram-positive bacterial cells. Preferably, the bacterial cells or heterologous host cells are of the phylum Actinobacteria, more preferably of the class Actinomycetes, order Actinomycetales or family Actinomycetaceae. Preferably, the bacterial cells or heterologous host cells are of the genus Streptomyces.

Preferably, the positive regulatory gene is obtained from or derived from the same genus, species or strain as the bacterial cell.

Preferably, the positive regulatory gene is selected from the group consisting of the LuxR family of genes, SARP (Streptomyces antibiotic regulatory protein) genes and AraC genes. Most preferably, the positive regulatory gene is the LAL gene.

Preferably, when expressed, the positive regulatory gene is operably-associated with a heterologous promoter, preferably wherein the heterologous promoter is:
(i) an inducible promoter,
(ii) a constitutive promoter, or
(iii) a growth-phase dependent promoter.

Preferably, when expressed, the nucleotide sequence coding for the positive regulatory gene is codon-altered compared to the wild-type nucleotide sequence of the positive regulatory gene.

Preferably, when expressed, the G+C content of the nucleotide sequence of the positive regulatory gene has been reduced compared to the G+C content of the wild-type nucleotide sequence of the positive regulatory gene.

Preferably, when expressed, the G+C content of the nucleotide sequence of the positive regulatory gene is less than 70%, more preferably less than 65%.

Preferably, the chemical entity is a product resulting from the expression of a biosynthetic gene cluster.

Preferably, the chemical entity is a polyketide, non-ribosomal peptide, terpene or RiPP.

The invention also provides a LAL gene having a G+C content of less than 70%, e.g. 55-65%, 55-60% or 60-65%; and a process for producing a modified bacterial cell, the process comprising the step of deleting a LAL gene or a LAL-regulator binding site from the genome of a cell, preferably a cell of the phylum Actinobacteria.

A “positive regulatory gene” is a gene which is involved in promoting the expression of one or more other genes. Preferably, the positive regulatory gene encodes a DNA-binding polypeptide. When this DNA-binding polypeptide is expressed, it leads to the upregulation of the one or more other genes. In the context of this invention, the other genes are ones which are in a biosynthetic gene cluster.

Preferably, therefore, the positive regulatory gene is a gene which encodes a DNA-binding polypeptide which, when expressed, leads to the upregulation of one or more of the genes in a biosynthetic cluster. Preferably, the biosynthetic cluster is one which is in the proximity of the positive regulatory gene in the cell's genome.

Examples of positive regulatory genes include the LuxR family of genes, SARP (Streptomyces antibiotic regulatory protein) genes and AraC genes.

LuxR regulators are a widely-studied group of bacterial helix-turn-helix (HTH) transcription factors involved in the regulation of many genes coding for important traits at an ecological and medical level. This regulatory family is particularly known by their involvement in quorum-sensing (QS) mechanisms, i.e. in the bacterial ability to communicate through the synthesis and binding of molecular signals (Lopes Santos et al., PLOS ONE, 1 Oct. 2012, volume 7, Issue 10, e46758).

Preferably, the positive regulatory gene is a large ATP-binding regulator of the LuxR family (i.e. LAL) gene.

Preferably, the positive regulatory gene is from or derived from the same genus, species or strain as the bacterial cell.

The large ATP-binding LuxR-like (LAL) family of transcriptional regulators are proposed to function as pathway-specific activators of some biosynthetic gene clusters. The LAL protein contains a N-terminal ATPase domain and a C-terminal LuxR family DNA-binding domain with a helix-turn-helix motif. LAL homologues have been shown to activate the production of several actinomycete-specialised metabolites, including pikromycin (PikD) (Wilson et al., 2001), rapamycin (RapH) (Kuscer et al., 2007) and the stambomycins (SAMR0484) (Laureti et al., 2011).

Numerous LAL nucleotide and amino acid sequences are known in the art, including pikD pikromycin AAC68887.1, nysRI nystatin AAF71778.1, mysRII nystatin AAF71779.1, nyrsrRIII nystatin AAF71780.1, samr0484 stambomycin CAJ88194.1, totR1 totopotensamide ATL73051.1, totR2 totopotensamide ATL73052.1, totR4 totopotensamide ATL73056.1 and vemR venemycin QAT18848.1.

The LAL sequences are diverse in nature, with sequence identities as low as 40% between LAL genes from different organisms.

As used herein, the term “LAL gene” preferably includes, but is not limited to:
(a) a polynucleotide molecule whose nucleotide sequence comprises or consists of the nucleotide sequence given in SEQ ID NO: 1;
(b) a polynucleotide molecule whose nucleotide sequence comprises or consists of a variant of the nucleotide sequence given in SEQ ID NO: 1, the variant having at least 30%, 40%, 50%, 60%, 70%, 80%, 85%, 90%, 95% or 99% sequence identity to SEQ ID NO: 1; and
(c) a polynucleotide molecule whose nucleotide sequence comprises or consists of a nucleotide sequence which encodes:
   (i) a polypeptide whose amino acid sequence is given in SEQ ID NO: 2, or
   (ii) a variant of (i), the variant having at least 30%,40%, 50%, 60%, 70%, 80%, 85%, 90%, 95% or 99% sequence identity to SEQ ID NO: 2.

Preferably, the variant is or encodes a transcriptional regulator comprising a N-terminal ATPase domain and a C-terminal LuxR family DNA-binding domain with a helix-turn-helix motif.

As used herein, the term “LAL polypeptide” refers to a polypeptide whose amino acid sequence comprises or consists of the amino sequence as given in SEQ ID NO: 2, or variant thereof having at least 30%, 40%, 50%, 60%, 70%, 80%, 85%, 90%, 95% or 99% sequence identity thereto.

Preferably, the variant encodes a transcriptional regulator comprising a N-terminal ATPase domain and a C-terminal LuxR family DNA-binding domain with a helix-turn-helix motif.

The LAL polypeptide comprises a N-terminal ATPase domain and a C-terminal LuxR family DNA-binding domain with a helix-turn-helix motif.

As used herein, the term “N-terminal ATPase domain” preferably includes, but is not limited to:
(a) a polynucleotide molecule whose nucleotide sequence comprises or consists of the nucleotide sequence given in SEQ ID NO: 3;
(b) a polynucleotide molecule whose nucleotide sequence comprises or consists of a variant of the nucleotide sequence given in SEQ ID NO: 3, the variant having at least 30%, 40%, 45%, 50%, 60%, 70%, 80%, 85%, 90%, 95% or 99% sequence identity to SEQ ID NO: 3; and
(c) a polynucleotide molecule whose nucleotide sequence comprises or consists of a nucleotide sequence which encodes:
   (i) a polypeptide whose amino acid sequence is given in SEQ ID NO: 4, or
   (ii) a variant of (i), the variant having at least 45%, 50%, 60%, 70%, 80%, 85%, 90%, 95% or 99% sequence identity to SEQ ID NO: 4.
      Preferably, the variant is or encodes an ATPase domain.

As used herein, the term “N-terminal ATPase domain” refers to a polypeptide whose amino acid sequence comprises or consists of the amino sequence as given in SEQ ID NO: 4, or variant thereof having at least 45%, 50%, 60%, 70%, 80%, 85%, 90%, 95% or 99% sequence identity thereto. Preferably, the variant encodes an ATPase domain.

As used herein, the term “C-terminal LuxR family DNA-binding domain with a helix-turn-helix motif” preferably includes, but is not limited to:
(a) a polynucleotide molecule whose nucleotide sequence comprises or consists of the nucleotide sequence given in SEQ ID NO: 5;
(b) a polynucleotide molecule whose nucleotide sequence comprises or consists of a variant of the nucleotide sequence given in SEQ ID NO: 5, the variant having at least 50%, 60%, 70%, 80%, 85%, 90%, 95% or 99% sequence identity to SEQ ID NO: 5; and
(c) a polynucleotide molecule whose nucleotide sequence comprises or consists of a nucleotide sequence which encodes:
   (i) a polypeptide whose amino acid sequence is given in SEQ ID NO: 6, or
   (ii) a variant of (i), the variant having at least 70%, 80%, 85%, 90%, 95% or 99% sequence identity to SEQ ID NO: 6.
      Preferably, the variant is or encodes a DNA-binding domain with a helix-turn-helix motif.

As used herein, the term “C-terminal LuxR family DNA-binding domain with a helix-turn-helix motif” refers to a polypeptide whose amino acid sequence comprises or consists of the amino sequence as given in SEQ ID NO: 6, or variant thereof having at least 70%, 80%, 85%, 90%, 95% or 99% sequence identity thereto. Preferably, the variant encodes a DNA-binding domain with a helix-turn-helix motif.

Preferably, the term “N-terminal ATPase domain” includes, but is not limited to:
(a) a polynucleotide molecule whose nucleotide sequence comprises or consists of the nucleotide sequence given in SEQ ID NO: 3;
(b) a polynucleotide molecule whose nucleotide sequence comprises or consists of a variant of the nucleotide sequence given in SEQ ID NO: 3, the variant having at least 40% or 50% sequence identity to SEQ ID NO: 3; and
(c) a polynucleotide molecule whose nucleotide sequence comprises or consists of a nucleotide sequence which encodes:
   (i) a polypeptide whose amino acid sequence is given in SEQ ID NO: 4, or
   (ii) a variant of (i), the variant having at least 50% or 60% sequence identity to SEQ ID NO: 4,
      and the term “C-terminal LuxR family DNA-binding domain with a helix-turn-helix motif” preferably includes, but is not limited to:
(a) a polynucleotide molecule whose nucleotide sequence comprises or consists of the nucleotide sequence given in SEQ ID NO: 5;
(b) a polynucleotide molecule whose nucleotide sequence comprises or consists of a variant of the nucleotide sequence given in SEQ ID NO: 5, the variant having at least 60% or 70% sequence identity to SEQ ID NO: 5; and
(c) a polynucleotide molecule whose nucleotide sequence comprises or consists of a nucleotide sequence which encodes:
   (i) a polypeptide whose amino acid sequence is given in SEQ ID NO: 6, or
   (ii) a variant of (i), the variant having at least 70% or 80% sequence identity to SEQ ID NO: 6.

There are many established algorithms available to align two amino acid or nucleic acid sequences. Typically, one sequence acts as a reference sequence, to which test sequences may be compared. The sequence comparison algorithm calculates the percentage sequence identity for the test sequence(s) relative to the reference sequence, based on the designated program parameters. Alignment of amino acid or nucleic acid sequences for comparison may be conducted, for example, by computer-implemented algorithms (e.g. GAP, BESTFIT, FASTA or TFASTA), or BLAST and BLAST 2.0 algorithms.

Percentage amino acid sequence identities and nucleotide sequence identities may be obtained using the BLAST methods of alignment (Altschul et al. (1997), “Gapped BLAST and PSI-BLAST: a new generation of protein database search programs”, Nucleic Acids Res. 25:3389-3402; and http://www.ncbi.nlm.nih.gov/BLAST). Preferably the standard or default alignment parameters are used.

Standard protein-protein BLAST (blastp) may be used for finding similar sequences in protein databases. Like other BLAST programs, blastp is designed to find local regions of similarity. When sequence similarity spans the whole sequence, blastp will also report a global alignment, which is the preferred result for protein identification purposes.

Preferably the standard or default alignment parameters are used. In some instances, the “low complexity filter” may be taken off.

BLAST protein searches may also be performed with the BLASTX program, score=50, wordlength=3. To obtain gapped alignments for comparison purposes, Gapped BLAST (in BLAST 2.0) can be utilized as described in Altschul et al. (1997) Nucleic Acids Res. 25: 3389. Alternatively, PSI-BLAST (in BLAST 2.0) can be used to perform an iterated search that detects distant relationships between molecules. (See Altschul et al. (1997) supra). When utilizing BLAST, Gapped BLAST, PSI-BLAST, the default parameters of the respective programs may be used.

With regard to nucleotide sequence comparisons, MEGABLAST, discontiguous-megablast, and blastn may be used to accomplish this goal. Preferably the standard or default alignment parameters are used. MEGABLAST is specifically designed to efficiently find long alignments between very similar sequences. Discontiguous MEGABLAST may be used to find nucleotide sequences which are similar, but not identical, to the nucleic acids of the invention.

The BLAST nucleotide algorithm finds similar sequences by breaking the query into short subsequences called words. The program identifies the exact matches to the query words first (word hits). The BLAST program then extends these word hits in multiple steps to generate the final gapped alignments. In some embodiments, the BLAST nucleotide searches can be performed with the BLASTN program, score=100, wordlength=12.

One of the important parameters governing the sensitivity of BLAST searches is the word size. The most important reason that blastn is more sensitive than MEGABLAST is that it uses a shorter default word size (11). Because of this, blastn is better than MEGABLAST at finding alignments to related nucleotide sequences from other organisms. The word size is adjustable in blastn and can be reduced from the default value to a minimum of 7 to increase search sensitivity.

A more sensitive search can be achieved by using the newly-introduced discontiguous megablast page (www.ncbi.nlm.nih.gov/Web/Newsltr/FallWinter02/blastlab.html). This page uses an algorithm which is similar to that reported by Ma et al. (Bioinformatics. 2002 March; 18(3): 440-5). Rather than requiring exact word matches as seeds for alignment extension, discontiguous megablast uses non-contiguous word within a longer window of template. In coding mode, the third base wobbling is taken into consideration by focusing on finding matches at the first and second codon positions while ignoring the mismatches in the third position. Searching in discontiguous MEGABLAST using the same word size is more sensitive and efficient than standard blastn using the same word size. Parameters unique for discontiguous megablast are: word size: 11 or 12; template: 16, 18, or 21; template type: coding (0), non-coding (1), or both (2).

In some embodiments, the BLASTP 2.5.0+ algorithm may be used (such as that available from the NCBI) using the default parameters.

In other embodiments, a BLAST Global Alignment program may be used (such as that available from the NCBI) using a Needleman-Wunsch alignment of two protein sequences with the gap costs: Existence 11 and Extension 1.

Some aspects of the invention involve screening for the presence of a chemical entity or a biosynthetic gene cluster in a bacterial cell. The bacterial cells may be Gram-positive or Gram-negative cells. Preferably, the bacterial cells are Gram-positive. More preferably, the bacterial cells are of the phylum Actinobacteria.

Actinobacteria are a large phylum consisting of over 350 genera. Although most research on Actinobacteria has focused on the Streptomyces genus, many rarer actinobacterial genera (e.g. Salinispora, Amycolatopsis and Micromonospora) also produce structurally-diverse natural products.

Preferably, the bacterial cells are of the class Actinomycetes. Preferably, the bacterial cells are of the order Actinomycetales. Preferably, the bacterial cells are of the family Actinomycetaceae . Preferably, the bacterial cells are of the genus Streptomyces.

Examples of Streptomyces species include Streptomyces coelicolor A3(2), Streptomyces ambofaciens, Streptomyces scabies and Streptomyces venezuelae.

The development of numerous low-cost, high-throughput sequencing methods has lead to a vast increase in the availability of genomic data from public databases such as the National Center for Biotechnology Information (NCBI). Numerous computational tools may be used to screen this genomic data (e.g. BLAST). Thus in one embodiment, the “identifying” in Step (a) is carried out by comparing the nucleotide or amino acid sequence of the positive regulatory gene against the corresponding sequence of the genome of the bacterial cell.

Whilst standard screening methods may be used in the context of this invention, the low levels of sequence identity between known LAL genes means that finding unknown LAL genes within genomic data can be problematic. Preferably, therefore, steps are taken to increase the chances of finding unknown LAL genes. Such steps include, for example, manually annotating the bacterial genomes; and searching for sequence homology between a number of positive regulatory genes (e.g. a number of LAL genes) and bacterial genomes.

Most preferably, the location of a nucleotide sequence coding for a LAL gene within the nucleotide sequence of the genome of the cells is determined using a Hidden Markov model. For example, a Hidden Markov model may be created (using readily available software), e.g. using a sequence alignment from a plurality of positive regulatory genes (e.g. 15-25 genes).

Some aspects of the invention involve the step of analysing the nucleotide sequence of the cell genome which is in the proximity of the location of the nucleotide sequence of the positive regulatory (e.g. LAL) gene in order to determine the presence (or absence) of a nucleotide sequence which codes for a biosynthetic gene cluster.

Biosynthetic gene clusters are often found in the proximity of positive regulatory genes (e.g. LAL genes). The positive regulatory genes (e.g. LAL genes) may be found at any position in relation to the biosynthetic gene cluster, e.g. at the 5′ end, within the cluster or at the 3′ end.

As used herein, the term “in the proximity of” refers to a distance of less than 250 Kb, less than 150 Kb, less than 100 Kb or less than 50 Kb, wherein the distance is measured from the start codon of the positive regulatory gene (e.g. LAL gene) to the start codon of the closest biosynthetic gene in the cluster.

It is often the case that positive regulatory genes (e.g. LAL genes) are found within the biosynthetic gene cluster. In some cases, other (non-cluster) genes may be present between the positive regulatory gene (e.g. LAL gene) and the cluster.

A biosynthetic gene cluster encodes all of the proteins needed to assemble a specialised metabolite from primary cellular metabolites. The actinorhodin biosynthetic gene cluster in Streptomyces coelicolor A3(2) is an archetypal example. As used herein, therefore, the term “biosynthetic gene cluster” (BGC) refers to a stretch of DNA which comprises a group of genes involved in the production of one or more specific compounds. The genes may all encode different proteins. These specific compounds may be referred to interchangeably herein as the “metabolite”, “natural product”, “end product” or “product resulting from the expression of the bacterial gene cluster”.

Some of the genes within the cluster may code for enzymes. Other genes may code for polypeptides which may serve simply to carry intermediates and do not have an explicit catalytic function. Others genes may code for regulators that bind DNA, or efflux pumps that confer self resistance.

The cluster may comprise two or more genes, e.g. 2-70 genes, or at least 3, 4, 5, 6, 7, 8, 9,10, 20, 30, 40 or 50 genes. The stretch of DNA may span 2-250 Kb, e.g. 10-100 or 10-50 Kb.

Preferably, the BGC is a cryptic BGC. A cryptic biosynthetic gene cluster is one for which the metabolic product(s) are not known. Preferably, the BGC is a silent BGC. A silent biosynthetic gene cluster is one that is not expressed (or expressed so weakly that the metabolic product is difficult to detect using standard procedures) in standard laboratory cultures.

There are numerous tools which are available for the analysis of nucleotide sequences for determining whether a biosynthetic gene cluster is present (see for example, http://www.secondarymetabolites.org/mining/; and Weber and Kim, Synthetic and Systems Biotechnology, Volume 1, Issue 2, June 2016, pages 69-79).

In the first step of the analysis, genes which encode conserved enzymes or protein domains that have known roles in secondary metabolism are identified in the proximity of the positive regulatory gene, for example, the “condensation (C)”, “adenylation (A)” and “peptidyl carrier protein (PCP)” domains of non-ribosomal peptide synthetases (NRPSs).

In the second step of the analysis, predefined rules may be used to associate the presence of such conserved enzymes or protein domains with defined classes of natural products.

For example, a NRPS biosynthetic gene cluster can be simply and unambiguously identified if genes are present that code for at least one C-, A- and PCP domain.

More complex rules may then be used to take into account whether specific genes are encoded in close proximity; for example, type II polyketide BGCs can be detected using a rule that evaluates whether a ketosynthase α, a ketosynthase β/chain length factor and acyl-carrier protein are encoded by three individual genes in direct proximity.

These approaches can be very precise in detecting gene clusters of known families and classes of which rules can be defined. Based on the prerequisite to have defined rules, these algorithms cannot detect novel pathways that use a different biochemistry and enzymes.

Rule-independent methods, which are less biased towards known clusters, have also been developed. These tools use machine learning-based approaches or automated phylogenomics analyses to make their predictions.

The tools which are available for the analysis of biosynthetic gene clusters include:
2metDB, antiSMASH, ARTS, BAGEL, BiG-SCAPE, CASSIS and SMIPS, CLUSEAN, ClusterFinder, ClusterTools, ClustScan Professional, eSNaPD//environmental, Surveyor of Natural Product Diversity, EvoMining, FunGeneClusterS, MIDDAS-M, MIPS-CG, NaPDoS//Natural Products Domain Seeker, PhytoClust, PKMiner, plantiSMASH, PRISM/GNP, RiPPMiner, RODEO, SANDPUMA, SBSPKS, SeMPI and SMURF/Secondary Metabolite Unknown Region Finder.

In particular, computational tools like antiSMASH have played a central role through the analysis of Biosynthetic Gene Clusters (BGCs) in recent years. Thousands of candidate BGCs have thus been identified using computational tools such as antiSMASH (Blin et al., 2019) and ClusterFinder (Cimermancic et al. 2014). Databases like IMG-ABC (Hadjithomas et al., 2017) and antiSMASH-DB (Blin, Pascal et al., 2019) store many thousands of such computationally-predicted BGCs, potentially coding for a very diverse range of natural product classes. MIBiG 2.0 is a further repository for biosynthetic gene clusters of known function (Kautsar et al., 2020).

In some key embodiments of the invention, the intention of the method is to discover new biosynthetic clusters and/or new end products thereof. It is desirable therefore to include a step in the method by which known clusters are removed, discarded or ignored. This can be done, for example, using antiSMASH or by the manual annotation of the BGCs.

In some embodiments, therefore, the method of the invention comprises the step of removing or discarding biosynthetic clusters whose end product is already known.

In some embodiments of the invention, the method comprises the step of:
(c) proposing the molecular structure of a product resulting from the expression of the biosynthetic gene cluster.

As used here, the term “proposing the molecular structure” includes modelling, attempting to predict, predicting, and postulating a molecular structure for the product.

Once a new biosynthetic gene cluster has been identified, its sequence may be compared to sequences of known biosynthetic gene clusters in order to try to predict structural features of the metabolic product of the new biosynthetic gene cluster. Comparison of these structural features with the structures of known natural products in databases such as the Dictionary of Natural Products, NPAtlas or Scifinder enables the structural similarity of the predicted metabolic product of the biosynthetic gene cluster to known compounds to determined. For biosynthetic gene clusters encoding megasyntha(ta)ses (e.g. modular PKSs, NRPSs), the building blocks and functionality incorporated by each module can be predicted on the basis of comparative sequence analyses. This allows the prediction of the core scaffold of the metabolic product which is assembled by a given BGC. Identification of the enzymes encoded by the BGC give further indications of what functionalization of the core scaffold might take place during/after assembly.

In order to increase or induce expression of the BGC in the bacterial cell, the positive regulatory gene (e.g. LAL gene), or a derivative thereof, is expressed in the bacterial cell or the heterologous host cell.

In some embodiments of the invention, the positive regulatory gene (e.g. LAL gene), or derivative thereof, is expressed in the bacterial cell or the heterologous host cell by introducing into the cell a nucleic acid molecule (e.g. a vector or plasmid) whose nucleotide sequence comprises (i) a promoter, operably-associated with (ii) a nucleotide sequence coding for the positive regulatory gene or derivative thereof.

The positive regulatory gene (e.g. LAL gene) may be expressed as part of the BGC. Preferably, the positive regulatory gene (e.g. LAL gene) is expressed in the cell independently of the expression of the BGC.

The nucleic acid molecule (e.g. vector or plasmid) may be introduced into the cell such that the nucleic acid molecule becomes (i) stably integrated into the genome of the cell, or (ii) present episomally within the cell.

Preferably, the promoter which is operably-associated with the nucleotide sequence coding for the positive regulatory gene is a heterologous promoter (i.e. a promoter with which the positive regulatory gene is not naturally associated).

In some embodiments, the promoter is a constitutive promoter. Examples of constitutive promoters include ermE* gapdh(EL), rpsl(RO), and kasO*. Preferably, the constitutive promoter is the ermE* promoter.

In other embodiments, the promoter is an inducible promoter. Inducible promoter systems facilitate the control of the onset of metabolite production. This facilitates the storage of engineered bacteria and increase titres of new metabolites. Examples of inducible promoters include TetR/tetO, PnitA-NitR, OtrR, tipA, and mmfR/mmyR promoters. Preferably, the inducible promoter is the mmfR/mmyR promoter.

In other embodiments, the promoter is a growth-phase dependent promoter. Preferably, the growth-phase dependent promoter is the actll-orf4 promoter.

The nucleotide sequence of the positive regulatory gene may or may not be based on or derived from the nucleotide sequence of the positive regulatory gene which is present in the genome of the bacterial cell or heterologous host cell in which it is being expressed.

In embodiments of the invention wherein the positive regulatory gene is a LAL gene, the LAL gene is preferably one as defined herein.

In some embodiments, the vector or plasmid comprises:
(i) a promoter, operably-associated with
(ii) a nucleotide sequence coding for a positive regulatory gene (e.g. LAL gene), and
(iii) a repressor element.

The repressor element is located in the vector or plasmid such that the binding of a repressor to the repressor element represses transcription of the positive regulatory gene (e.g. LAL) gene. Examples of repressor elements and repressors include MmfR, TetR, ArpA, GbnR, ScbR AvaR1, and their associated operators. In some embodiments, the repressor is the MmfR repressor from S. coelicolor. MmfR may be released from its operator by 2-alkyl-4-hydroxymethylfuran-3-carboxylic acid (AHFCA).

The positive regulatory gene (e.g. LAL gene) may also be operably-associated with a suitable terminator, e.g. the fd terminator.

Actinobacteria have a high guanine+cytosine (G+C) content in their DNA. For example, the G+C content of some Actinobacteria can be as high as 70%.

This high G+C content creates problems with PCR amplification of Actinobacterial genes and the cloning of such genes. It also creates problems with the synthesis of such genes.

In order to address these issues, in embodiments of the invention wherein Actinobacterial genes (or other genes having high G+C contents) or nucleotide molecules derived therefrom are used, it is preferable to artificially lower the G+C content of the positive regulatory gene (e.g. LAL gene).

The inventors have found that using G+C-lowered LAL genes increases the transformation efficiency of such genes into cells, thus facilitating genetic engineering of those cells. In one embodiment, therefore, the invention provides a codon-altered positive regulatory gene (e.g. LAL gene) having a G+C content of less than 70%, e.g. 55-65%, 55-60% or 60-65%. This codon-altered positive regulatory gene (e.g. LAL gene) may be used in any of the vectors or plasmids described herein. As used herein, the term “positive regulatory gene or derivative thereof” includes codon altered genes.

Some wild-type LAL genes comprise a TTA codon. Expression of the tRNA that recognises the TTA codon is developmentally regulated, meaning its abundance is low until the cells are in stationary phase. In some embodiments, therefore, the LAL gene is one which does not comprise a TTA codon. For example, the TTA codon may be mutated to a TTG, CTT, CTC, CTA, or CTG codon. As used herein, the term “positive regulatory gene or derivative thereof” includes genes which do not comprise a TTA codon.

If the presence of a biosynthetic gene cluster is determined, then some aspects of the invention involve obtaining a nucleic acid molecule whose nucleotide sequence comprises the nucleotide sequence of the biosynthetic gene cluster.

In this regard, the term “obtaining” includes (i) synthesizing a nucleic acid molecule whose nucleotide sequence comprises the nucleotide sequence of the biosynthetic gene cluster (e.g. using standard DNA synthesis methods); and (ii) cloning a nucleic acid molecule whose nucleotide sequence comprises the nucleotide sequence of the biosynthetic gene cluster (e.g. from a cell of the organism in which the cluster was found).

For example, entire BGCs may be cloned directly from Actinobacterial genomes using transformation-associated recombination (TAR) in yeast. This facilitates co-expression of BGCs and LAL genes in a heterologous host. In other embodiments, BGCs may be cloned into E. coli-Streptomyces shuttle vectors. This facilitates their introduction into a wide range of Actinobacteria via intergenic conjugation.

Some aspects of the invention involve expressing a nucleic acid molecule whose nucleotide sequence comprises the nucleotide sequence of all or part of the biosynthetic gene cluster in a heterologous host cell. Several Actinobacteria have previously been employed in the art as heterologous hosts. Preferably, the host cell is a Streptomyces spp.

In particular, Streptomyces coelicolor M1152, Streptomyces avermitilis SUKA17 and Streptomyces albus J1704 have previously been rationally engineered to create ‘super hosts’. These have had potentially-competing pathways and/or highly-expressed BGCs removed, to facilitate high product titres from heterologously-expressed BGCs. Other suitable host cells include S. venezuelae, S. cinnamonensis C730.1 and C730.7, S. ambofaciens, S. roseosporus, S. fradiae and S. toyocaensis; and Streptomyces lividans TK23, TK24 and derivatives thereof. Further suitable hosts include Amycolatopsis japonicum, Saccharopolyspora erythrea and Salinispora tropica. Additional suitable hosts are described in Nat. Prod. Rep., (2019), 36, 1281-1294 (the contents of which is specifically incorporated herein by reference). In some embodiments, the host cell is Streptomyces fungicidicus or Streptomyces caelestis. The heterologous host cells may also be any of the bacterial cells as defined herein.

In some embodiments, the heterologous host cells are recombinant host cells.

Some potential host cells (bacterial cells or heterologous host cells) may comprise endogenous genes encoding positive regulatory gene (e.g. LAL) homologues and/or the operators to which positive regulatory gene (e.g. LAL) regulators bind. In such cases, it is preferable to delete such endogenous genes/operators, in order to prevent undesirable off-target interactions.

In some embodiments of the invention, therefore, the bacterial cell or heterologous host cell is one from which endogenous positive regulatory gene (e.g. LAL genes) or homologues thereof have been deleted; and/or one from which endogenous positive (e.g. LAL) regulator binding sites have been deleted.

The invention also provides a process for producing a modified bacterial cell, the process comprising the step of deleting a LAL gene or a LAL-regulator binding site from the genome of a cell, preferably a cell of the phylum Actinobacteria. In some preferred embodiments of this aspect of the invention, the cell is Streptomyces fungicidicus, Streptomyces caelestis or Saccarapolyspora spinosa.

The invention also provides a host cell from which endogenous positive regulatory gene (LAL genes) or homologues thereof have been deleted; and/or from which endogenous positive (e.g. LAL) regulator binding sites have been deleted.

Some potential host cells may comprise BGCs encoding pathways that could compete for precursors with the heterologous (newly-introduced) BGCs. In such cases, it is preferable to delete such BGCs in order maximize metabolic fluxes through the heterologous (newly-introduced) pathways. This also simplifies the metabolite profiling and identification of any products of the BGC. In some embodiments of the invention, therefore, the cell is one from which all or part of a BGC has been deleted. In some preferred embodiments of this aspect of the invention, the host cell is Streptomyces fungicidicus, Streptomyces caelestis or Saccarapolyspora spinosa.

When using a constitutive promoter to express the positive regulatory gene (e.g. LAL gene), it might be the case that initially high levels of metabolite production are lost with repeated passaging of the cells. This is probably because high levels of metabolite production slow growth, causing high-producing cells to be selected out of the cell population.

This can be a problem during the scaling-up of metabolite production to obtain sufficient material for biological testing.

One way to get around this is to use an inducible or growth-phase dependent promoter to control expression of the positive regulatory gene (e.g. LAL gene), as discussed above.

An alternative way to address this issue is to introduce or select for random mutations in the heterologous BGC which down-regulate expression of the BGC in the host cell. This step would be carried out prior to the expression in the host cell of the nucleic acid molecule whose nucleotide sequence comprises the nucleotide sequence of the biosynthetic cluster.

In yet a further embodiment, therefore, the invention provides the step of mutating the nucleotide sequence of the biosynthetic gene cluster in order to reduce the expression level of one or more of the genes in the biosynthetic gene cluster (compared to the expression level of the corresponding gene from the non-mutated cluster). The mutation step may be carried out in any standard manner.

In some embodiments, the method of the invention comprises isolating and/or identifying the chemical entity. In some embodiments, the method of the invention comprises isolating and/or identifying a product resulting from the expression of the biosynthetic gene cluster. Such chemical entities and products which result from the expression of the biosynthetic gene cluster may be isolated by any suitable technique. Many such techniques are known in the art, including chromatographic techniques such as HPLC, flash column chromatography, organic extraction, hydrophobic interaction chromatography, and ion exchange chromatography.

Products resulting from the expression of the biosynthetic gene cluster may be identified by any suitable technique. Many such techniques are known in the art, including LC-MS, NMR, MS-MS and HPLC.

For example, a high resolution Bruker MaXis Impact mass spectrometer coupled with a Dionex UHPLC system may be used, which is capable of handling 96 and 384-well plates. UHPLC offers much shorter run times and consumes far less material than conventional HPLC separations and the metabolomics software supplied with these instruments allows rapid comparison of metabolite profiles to identify new products resulting from the expression of the biosynthetic gene cluster.

In other embodiments, the method of the invention comprises determining the presence of one or more chemical entities, other than the polypeptide which is encoded by the positive regulatory gene (e.g. LAL gene), whose expression level is increased in the bacterial cell after the expression of the positive regulatory gene (e.g. LAL gene). Such chemical entities may also be isolated and identified using one or more of the techniques mentioned above.

Examples of chemical entities (i.e. metabolic products) which may be screened for include polyketides, non-ribosomal peptides, terpenes and ribosomally synthesised and post translationally modified peptides (RiPPs). In some embodiments, the chemical entity (i.e. metabolic product) is not a polyketide. In some embodiments, the chemical entity (i.e. metabolic product) is a non-ribosomal peptide, terpene or RiPP.

In yet a further embodiment, the invention provides a product produced by the expression of a biosynthetic cluster which has been found by a method of the invention.

Parts or all of some of the methods of the invention may be computer-implemented.

Preferably, the method steps are carried out in the order specified.

The nucleic acid molecules, vectors and plasmids used in the invention may be made by any suitable technique. Recombinant methods for the production of the nucleic acid molecules and host cells of the invention are well known in the art (e.g. “Molecular Cloning: A Laboratory Manual” (Fourth Edition), Green, M R and Sambrook, J., (updated 2014)).

The disclosure of each reference set forth herein is specifically incorporated herein by reference in its entirety.

### BRIEF DESCRIPTION OF THE FIGURES

FIG. 1: The conserved domain search output for SamR0484 (top) and the HMM created to search for LAL-encoding genes (bottom).

FIG. 2: Phylogenetic comparison of LALs highlighting proteins that are similar to those reported to regulate the biosynthesis of known natural products.

FIG. 3: Transformants of S. caelestis NRRL 2821 obtained using a plasmid containing native strvi_8009 (left) and a codon-altered derivative (right).

FIG. 4: Chromatograms from LC-MS analyses of culture extracts of S. rochei overexpressing a LAL regulator gene. A fresh transformant (top) produces novel metabolites that are absent in cultures grown from spore stocks of a transformant that has been stored for 4 weeks (bottom).

FIG. 5: Chromatograms from LC-MS analyses of culture extracts of S. caelestis NRRL 2821 wild type (top) and S. caelestis NRRL 2821 overexpressing codon-altered strvi_8009 (bottom). Peaks corresponding to the novel metabolites identified as the likely products of the BGC associated with strvi_8009 are highlighted (yellow band).

### EXAMPLES

The present invention is further illustrated by the following Examples, in which parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1: Screening for the Presence of LAL Genes in Actinobacterial Genomes

44 Actinobacteria were used to test different approaches to activate silent biosynthetic gene clusters (BGCs). The genome sequences of these organisms contained over 1,500 BGCs and we aimed to prioritize and activate those that were silent. An approach based around the large ATP-binding regulators of the LuxR (LAL) family proved to be generalizable.

### Identifying Novel LAL Regulator Genes

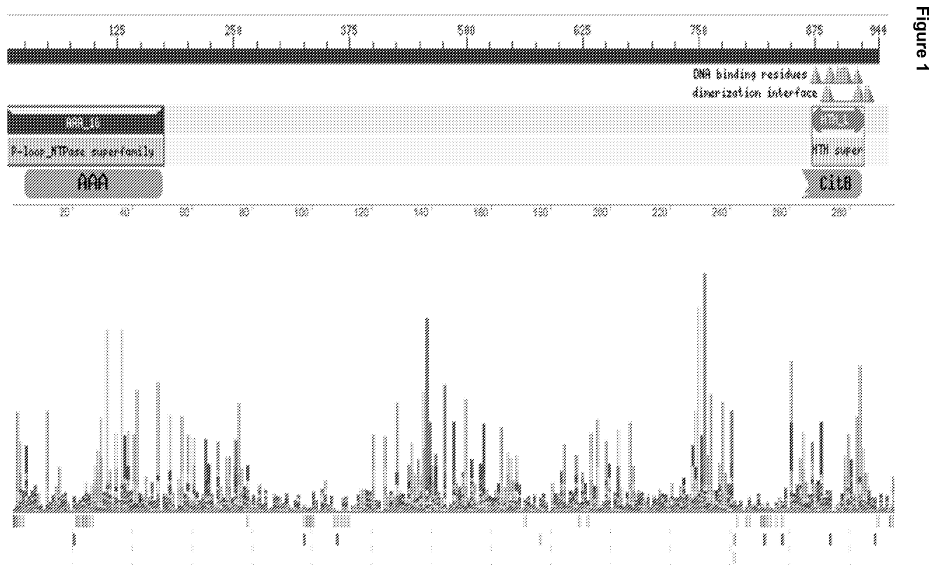

We used the gene encoding the LAL (samR0484 in Streptomyces ambofaciens) that regulates the expression of the stambomycin BGC to “BLAST” our collected genomes and found 18 genes encoding LAL regulators. We were able to find a larger number of LAL regulators genes in our collection of genomes when we began to manually annotate BGCs. LAL regulators contain a ATP-binding subdomain (at the N-terminus) and a DNA-binding subdomain (at the C-terminus) but the central 600 amino acids have no obvious subdomain structure (FIG. 1). This low sequence homology across the entire length of the protein made it difficult to discover LAL regulators through BLAST nucleotide searches alone.

A sequence alignment of 20 reported LAL regulators in the literature and the 18 regulators that we found within our strain collection was used to create a Hidden Markov Model (HMM) using HMMER 3.1b2 (Finn, R. D.; Clements, J.; Eddy, S. R. Nucleic Acids Res. 2011, gkr367) (FIG. 1). This HMM was used to search our collection of genomes and it found over 250 LAL regulator genes. We also used the HMM to search the NCBI non-redundant database where over 17,000 examples of LAL regulators genes were found.

Using an HMM to search genomes allowed us to find not only a greater number of LAL regulators genes, but also showed that these genes are associated with BGCs for a greater range of natural product classes (i.e. polyketides, non-ribosomal peptides, ribosomally-synthesized and post-translationally modified peptides, terpenes and non-canonical pathways) than had been previously observed.

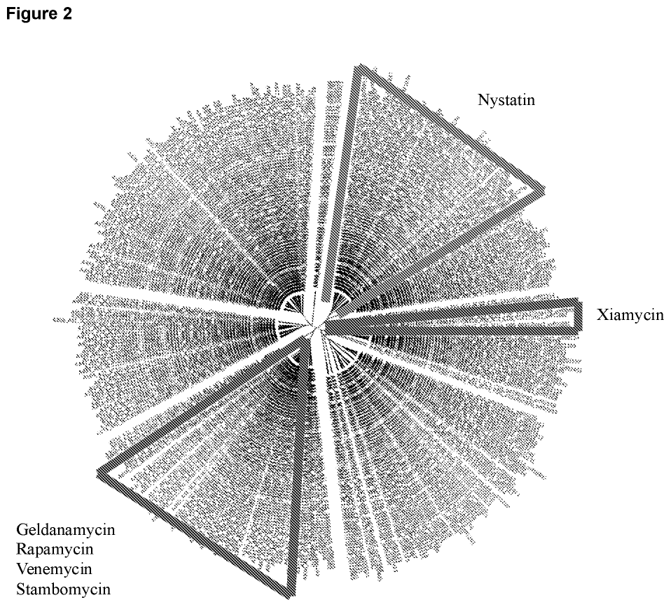

To prioritize which BGCs to focus on, phylogenetic methods were used to identify proteins that were similar to known LAL regulators (FIG. 2). Further bioinformatics analyses were used to predict structural features of the metabolic products of these BGCs enabling us to prioritise those likely to direct the assembly of novel compounds.

More specifically, phylogenetic analyses of particular enzymatic domains (e.g. KS and TE) were used to help distinguish which biosynthetic gene clusters would produce novel natural products. This was combined with manual annotation, which enabled the core scaffold of the unknown natural product to be predicted. This core scaffold can be searched against databases to establish its similarity to reported compounds.

### Example 2: Cloning/Synthesis of Novel LAL Regulator Genes

In S. caelestis NRRL 2821, we found a BGC that contained a LAL regulator gene that was predicted to direct the assembly of a novel non-ribosomal peptide. This metabolite was not detected when the wild-type strain was cultured. The process of manipulating the LAL regulator gene to activate expression of the BGC and discover its metabolic product is described below as an example of aspects of the invention.

To constitutively express LAL regulator genes, we first attempted to amplify them by PCR from genomic DNA and ligate the PCR product into a plasmid that will put this gene under the control of a constitutive promoter (e.g. ermE*). The gene (strvi_8009) encoding the LAL regulator that is proposed to control the BGC in S. caelestis NRRL 2821 contains 2703 base pairs and has a GC content of 70%. Although this GC content is lower than we typically observe for genes encoding LAL regulators (˜75%) in Actinobacteria, it proved challenging to amplify this gene using PCR. We eventually succeeded by using a high concentration of DMSO (10% v/v) in the reaction, but were only able to obtain the product in low quantities (<1 ng/μL). The PCR product was first cloned into a shuttle vector, then sub-cloned into a plasmid that places the gene under the control of the ermE* promoter.

Although it is possible to clone LAL regulators genes using traditional approaches, separate optimization is required for each regulator gene, which prevents the process from being scaled. To overcome this problem, we turned to gene synthesis. However, it also proved difficult to synthesize large genes (>3 kb) with high GC content. Initial efforts to synthesize three LAL genes with high native GC content failed. To overcome this, we codon-altered the genes to lower the GC-content to 55-65% across their length. The following parameters were applied:
GC content between 30-65%
No window of 100 bp with GC higher than 75%
No window of 50 bp with GC higher than 80%
No 16mer repeats
No TTA codons

Using these parameters, we were able to synthesize 25 refactored LAL genes, which were cloned into plasmids that placed them under the control of a constitutive promoter.

### Example 3: Transformation of Actinobacteria

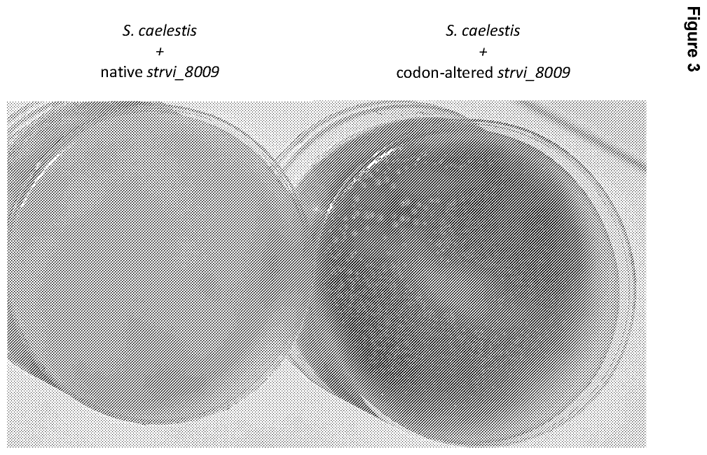

Genetic-engineering protocols for Actinobacteria are well established. Using these protocols we were unable to transform S. caelestis NRRL 2821 with the native strvi_8009 gene. When the same experiment was attempted with the codon-altered strvi_8009 derivative, transformants were obtained (FIG. 3). The reason for the increased transformation efficiency is not currently understood.

### Methods

### Transformation of Actinobacteria with Codon-Altered LAL Regulator Genes

E. coli ET12567/pU8008 was transformed separately via electroporation with an integrative plasmid containing the codon-altered and native strvi_8009 gene under the control of the ermE* promoter. The transformants were incubated for 1 hour at 37° C. and then spread on LB agar containing kanamycin (50 μg/mL of LB), chloramphenicol (50 μg/mL of LB) and ampicillin (100 μg/mL of LB). The resulting plated bacteria were incubated overnight at 37° C. A single colony was picked and grown in liquid LB medium containing kanamycin (50 μg/mL of LB), chloramphenicol (50 μg/mL of LB) and ampicillin (100 μg/mL of LB) overnight. 300 μL of the overnight culture was inoculated into 10 ml of LB liquid medium containing kanamycin (50 μg/mL of LB), chloramphenicol (50 μg/mL of LB) and ampicillin (100 μg/mL of LB) and was grown to an OD ˜0.6 (about 5 hours). Cells were pelleted (5 mins, 4000 rpm) and washed three times with ice cold LB medium, and then resuspended in LB medium(500 μL).

Spores of S. caelestis NRRL 2821 (100 μL) were heat shocked in TSB medium (500 μL) at 55° C. for 10 min and then incubated at 30° C. for 5 hours. The E. coli donor cells, prepared as described above, were gently combined with the S. caelestis culture and the mixture was pelleted (2 min, 6000 rpm). 500 μL of the supernatant was removed and the cells were resuspended in the remaining liquid. The resulting mixture was spread on two SFM agar plates (supplemented with MgCl₂, 100 μM) which were incubated overnight at 30° C. and then overlayed with appropriate antibiotics. After 4-7 days cultivation at 30° C. the number of transconjugants on each plate was assessed (FIG. 3).

### Growth, Extraction and Metabolite Analysis (Liquid Cultures)

20 A single transformant was selected and grown in pre-culture medium (TSB, 50 mL) for 2 days at 30° C. 500 μL of the pre-culture was used to inoculate 50 mL of each growth medium (a minimal medium, a natural medium and a rich medium) and the resulting cultures were grown for 7 days at 30° C. The cultures were acidified (to pH 4 with 2M HCl) and extracted with ethyl acetate (3×50 mL). The combined organics were dried over MgSO₄ and evaporated to dryness. The residue was redissolved in acetonitrile/water (50/50 v/v, 1 mL) and analyzed by UHPLC-ESI-Q-TOF-MS as outlined below.

### Growth, Extraction and Metabolite Analysis (Solid Cultures)

A single transformant was selected and streaked on ISP4 agar medium. After 7 days growth at 30° C., the spores were harvested and used to inoculate agar plates containing three different media (a minimal medium, a natural medium and a rich medium). After 7 days incubation at 30° C. the cultures were acidified (to pH 4 with 2M HCl) and extracted with acetonitrile (10 mL). The combined organics were dried over MgSO₄ and evaporated to dryness. The residue was redissolved in acetonitrile/water (50/50 v/v, 1 mL) and analyzed by UHPLC-ESI-Q-TOF-MS as outlined below.

### UHPLC-ESI-Q-TOF-MS Equipment and Analysis Conditions

Analyses were carried out using a Bruker MaXis Impact ESI-TOF-MS connected to a Dionex 3000 RS UHPLC instrument fitted with an Agilent Zorbax Eclipse Plus C18 column (100×2.1 mm, 1.8 μm, 25° C.). The flow rate was 0.2 mL/min and a gradient of 5-100% acetonitrile (with 0.1% formic acid) over 20 min was used as the eluent.

### Example 4: Metabolite Detection

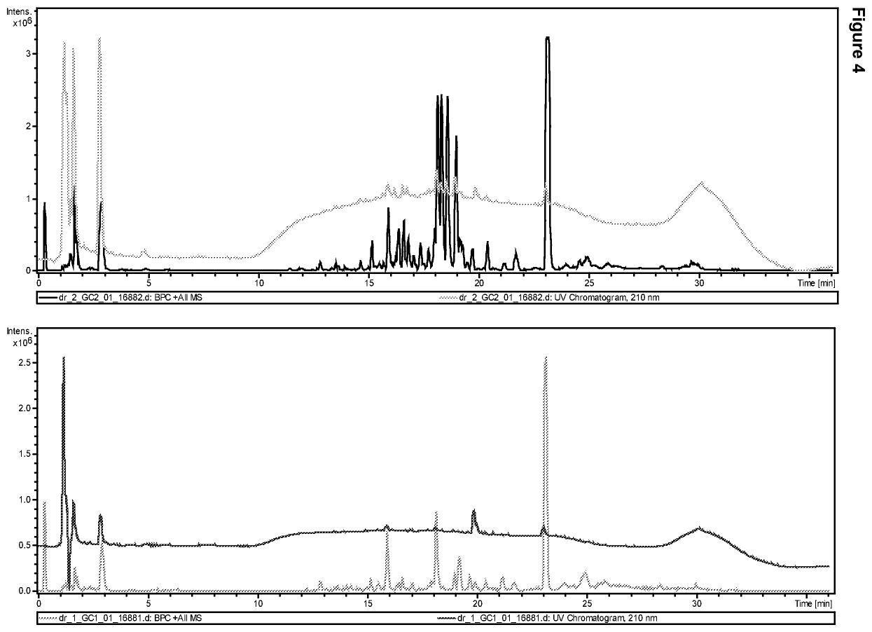

We previously found that transformants overexpressing LAL regulator genes would lose the ability to produce novel metabolites when stored (FIG. 4), hindering isolation and structure elucidation. To overcome this, we grew large-scale cultures directly from transformants, without producing an initial spore stock. We screened metabolite production in three different solid and liquid media (six in total).

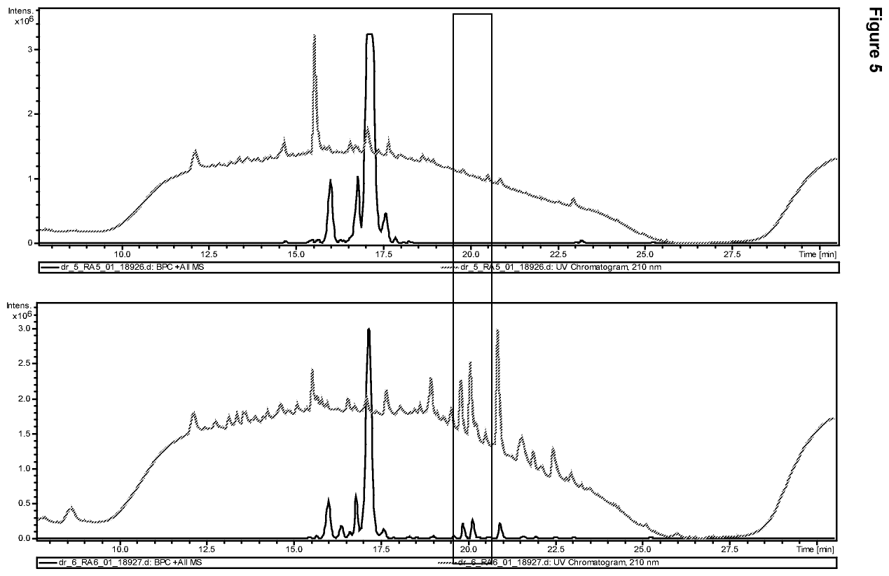

Ex-conjugants over-expressing strvi_8009 were screened for production of new metabolites. In rich media (both solid and liquid), the metabolite profile in the strain overexpressing the codon-altered strvi_8009 gene differed from the wild type strain (FIG. 5). Four novel compounds hypothesized to be the metabolic products of the BGC associated with strvi_8009 were identified.

### REFERENCES

Blin, K., Shaw, S., Steinke, K., Villebro, R., Ziemert, N., Lee, S. Y., Medema, M. H. and Weber, T. (2019) antiSMASH 5.0: updates to the secondary metabolite genome mining pipeline. Nucleic Acids Res., 47, W81-W87.
Blin, K., Pascal Andreu, V., de los Santos, E. L. C., Del Carratore, F., Lee, S. Y., Medema, M. H. and Weber,T. (2019) The antiSMASH database version 2: a comprehensive resource on secondary metabolite biosynthetic gene clusters. Nucleic Acids Res., 47, D625—D630.
Cimermancic, P., Medema, M. H., Claesen, J., Kurita, K., Wieland, Brown, L. C., Mavrommatis, K., Pati, A., Godfrey, P. A., Koehrsen, M., Clardy, J. et al. (2014) Insights into secondary metabolism from a global analysis of prokaryotic biosynthetic gene clusters. Cell, 158, 412-421.

Hadjithomas, M., Chen, I.-M. A., Chu, K., Huang, J., Ratner, A., Palaniappan, K., Andersen, E., Markowitz, V., Kyrpides, N. C. and Ivanova, N. N. (2017) IMG-ABC: new features for bacterial secondary metabolism analysis and targeted biosynthetic gene cluster discovery in thousands of microbial genomes. Nucleic Acids Res., 45, D560-D565.
Kautsar et al. D454—D458 Nucleic Acids Research, 2020, Vol. 48, Database issue Published online 15 October 2019 doi: 10.1093/nar/gkz882
E. Kuscer, N. Coates, I. Challis, M. Gregory, B. Wilkinson, R. Sheridan, H. Petkovic, J. Bacteriol. 2007, 189, 4756-4763.
Laureti, L. Song, S. Huang, C. Corre, P. Leblond, G. L. Challis, B. Aigle, Proc. Natl. Acad. Sci. USA 2011, 108, 6258-6263.
Thanapipatsiri et al., (2016) ChemBioChem 17, 2189-2198.
D. J. Wilson, Y. Xue, K. A. Reynolds, D. H. Sherman, J. Bacteriol. 2001, 183, 3468-3475.

### LIST OF SEQUENCES

The Sequence Listing filed with this patent application is fully incorporated herein as part of the description.

SEQ ID NO: 1

Samr0484 (stambomycin) nucleotide sequence.

SEQ ID NO: 2

Samr0484 (stambomycin) aa sequence.

SEQ ID NO: 3

N-terminal ATPase domain-encoding region from samr0484.

SEQ ID NO: 4

N-terminal ATPase domain from Samr0484.

SEQ ID NO: 5

C-terminal LuxR family DNA-binding domain-encoding region with a helix-turn-helix motif from samr0484.

SEQ ID NO: 6

C-terminal LuxR family DNA-binding domain with a helix-turn-helix motif from SamR0484.

1. A method for screening for the presence of a chemical entity in a bacterial cell, the method comprising the steps of:
(a) expressing a positive regulatory gene in a bacterial cell; and
(b) determining the presence of one or more chemical entities, other than the polypeptide which is encoded by the positive regulatory gene, whose expression level is increased in the bacterial cell after the expression of the positive regulatory gene, and optionally
(c) isolating and/or identifying the chemical entity.

2. A method for screening for the presence of a biosynthetic gene cluster in a bacterial cell, the method comprising the steps of:
(a) identifying the location of a nucleotide sequence coding for a positive regulatory gene within the nucleotide sequence of the genome of a bacterial cell; and
(b) analysing the nucleotide sequence of the cell genome in the proximity of the location of the nucleotide sequence of the identified positive regulatory gene in order to determine the presence of a nucleotide sequence which codes for a biosynthetic gene cluster;
optionally wherein the method is a computer-implemented method.

3. The method as claimed in claim 2, wherein the location of the nucleotide sequence coding for the positive regulatory gene is identified using a Hidden Markov model.

4. The method as claimed in claim 2, wherein the method additionally comprises the step of:
(c) proposing a molecular structure for a product resulting from the expression of the biosynthetic gene cluster.

5. The method as claimed in claim 2, wherein the method additionally comprises the steps of:
(d) obtaining a nucleic acid molecule whose nucleotide sequence comprises the nucleotide sequence of the biosynthetic gene cluster;
(e) expressing the nucleic acid molecule in a heterologous host cell;
(f) expressing the positive regulatory gene, or a derivative thereof, in the heterologous host cell; and optionally
(g) isolating and/or identifying a product resulting from the expression of the biosynthetic gene cluster.

6. A method for screening for the presence of a chemical entity in a bacterial cell, the method comprising the steps of:
(i) screening for the presence of a biosynthetic gene cluster in a bacterial cell, as claimed in claim 2; and
(ii) screening for the presence of a chemical entity in the bacterial cell by a method comprising the steps of:
(a) expressing a positive regulatory gene in a bacterial cell; and
(b) determining the presence of one or more chemical entities, other than the polypeptide which is encoded by the positive regulatory gene, whose expression level is increased in the bacterial cell after the expression of the positive regulatory gene, and optionally
(c) isolating and/or identifying the chemical entity;
wherein the biosynthetic gene cluster in Step (i) and (ii) are the same cluster; and the positive regulatory gene in Step (i) and (ii) are the same gene.

7. The method as claimed in claim 2, wherein the bacterial cell or heterologous host cell is a Gram-positive bacterial cell.

8. The method as claimed in claim 7, wherein the bacterial cell or heterologous host cell is of the phylum Actinobacteria.

9. The method as claimed in claim 8, wherein the bacterial cell or heterologous host cell is of the genus Streptomyces.

10. The method as claimed in claim 2, wherein the positive regulatory gene is obtained from or derived from the same genus, species or strain as the bacterial cell.

11. The method as claimed in claim 10, wherein the positive regulatory gene is selected from the group consisting of the LuxR family of genes, SARP (Streptomyces antibiotic regulatory protein) genes and AraC genes.

12. The method as claimed in claim 11, wherein the positive regulatory gene is the LAL gene.

13. The method as claimed in claim 2, wherein, when expressed, the positive regulatory gene is operably-associated with a heterologous promoter.

14. The method as claimed in claim 2, wherein, when expressed, the nucleotide sequence coding for the positive regulatory gene is codon-altered compared to the wild-type nucleotide sequence of the positive regulatory gene.

15. The method as claimed in claim 2, wherein, when expressed, the G+C content of the nucleotide sequence of the positive regulatory gene is reduced compared to the G+C content of the wild-type nucleotide sequence of the positive regulatory gene.

16. The method as claimed in claim 15, wherein, when expressed, the G+C content of the nucleotide sequence of the positive regulatory gene is less than 70%.

17. The method as claimed in claim 2, wherein the chemical entity is a product resulting from the expression of the biosynthetic gene cluster.

18. The method as claimed in claim 2, wherein the chemical entity is a polyketide, non-ribosomal peptide, terpene or RiPP.

19. A LAL gene having a G+C content of less than 70%.

20. A process for producing a modified bacterial cell, the process comprising the step of deleting a LAL gene or a LAL-regulator binding site from the genome of a cell.


SEQUENCE LISTING (6 sequences)

SEQ ID NO: 1 - DNA, 2877 bp, Streptomyces ambofaciens
atgctggtccatcgagacgaacaactggcccatctacgctgggcgttccacgcctgtgagaagcacggacacggacaggtggcgctggtcaccggcgctgtgggcagcggtaagacccaggtgctggagaccttcggggagtgggctgccaccgccggtgggcaggtgctgagcgccgccgggtcacgcgccgagcagggactccatttcggagtgctcgggcaactcctgcacagcgcgcggctcaaaccggaggggatccgggaggtcgaggagctgatacgcgaggtcgcgctggcggtgcccacggcggaggcccgaagcggcgcggcgcccgagaccttagccgaggccctgacagcggacgggctctgggcgccactgctgcggaccgccttcgacacgttcctgaccctggccggccgagggcctctggtgctggcggtcgacgacctccagcacacggacgccgcctcactgcactgtctgctctacgtcacccgaagactcagaaacgcacgcatcatggtgctgctctcggaggcgacgacgctgcgtccggcgcatccgctcttccacgcggagctgcgcagcctgcctcgcttcacccgggtcaccctgccgctcctcaccgccgactccgtctcccggctgctcggcgaggaggccgggacacccgacgtacgcggcgacggggccggggcaccggacgtacgcgaggaggccgggcggatcctgagggtgaccggcggcaatccgctgctgtcccaggcgctggtcgacgaacgggcccaccgcgggacggacagcgcccccgataccggagcgagggccggcgacaccttcgagagggccgtgctcgactgcctctatcgccacgagccgggcgttcgcctggtggcccaggcgctcgccgcgctggaccgcccggcctcaccggagctgctcggtcaactgctcgacgtgctgcccgactccaccgtgcccgccgtccgcgtcctcgagggcgcggggctcgtccacgcgggccggctgcgccatccacggatcgtgctcgcggtgcgttccgacatgccggccgaggaacggcggcggctgcaccagcgggcggccgaggtgttgcacgagaacggtgcggaagcgagcgtggtcgccgagcatctggtggcctcggcgtggacggacggtgcctgggtggtgcccgtcctccgggacgccgcggcgcacgcgctgtccaccggccgccccgaccacgccgccgcgtgtctgcgcctgggcggtcgagccgagaccgacaaggaccggcgcaactctctgatggcgatgctgatcaccgcccggtggcaggtgaatccgctggccgtgaccggtcaggtgagtcagttggtggaggcggcgcgggccgacgagtcgtccccttccacggccgtgtccgcggtgccgtacctgttgtggcagggacgggcggaggaggcggccgaggcgatcagcggttgtggtgcgggcgaccacggccccgcgtcgaacgagctgcggctgacccgcctgctgatcgcgctgtcccatccggactacctggggacggtacgggaggacccgagctcctggacgcgggcggcgacggcaccggactccgccagtccgctgctgcaggccgtgtcggtgctcggcaacgcgctgatgccgaccggcggggtggacaccgtggccgccgcggagcagctgctggagcggcatcatacggacagtggctccctgggtctgttgaccgcccccctcctggcgttgctctgtgccggccccgccgaccgggtcgcggtctggggcgaacggctgctggcacgacagggtgtccagcacacaccggcctggcggggtgtcatacgggcgatccacgcggaagcgacgctgcggctgggagacatggacgcggcggagcggggcgcgcggttggcgctggaggatctgcccgcgcctgcgtggggcgtggccgtcggtggtcccctgggcaccctgatcacctgcacgaccgagagtggccggctgttcgaggccgaacgctggctgtcgcagccggtaccggccggcgtcttccgcactccggtgggcgcccactatctgatcgcccggggccggcaccatctggccatgggacagcaccaggcggccgcggccgatctgcaccgcagcggtgagttggtccgttcctggggcatcgacgtggccggactggttccgtggcgcctggagctggcccgggtacagctcagcctcggccacagaacgcacgccgcgcaactgctgcaggagcagctgcaggtgtcccgcgggctggacgaccgtactcgcgggcgggcgctgcggctgctggcttccacggccacgccggttctgcggcgcaagctgctgtccaaggcggtgaccgtgttgcaggcatgcagtgaccagcaggagctggcccgggccctgtccgacacgagtcagacgctcccacagaccggtgacgccccacgggcccggctgttcgtacgtcgcgccggccggctggcacaggcctcgggccgggctccagcgctgccgcagcaggtgcagcgacggtccgcgcccagcgacgcggcgccgacggggggttcctcctcggatgcgtcttcggacggcttcgagtcccgggatcaggacggtctgctgagcgaggccgagcgaagggtcgcggtactggcggcacgggggcgtaccaaccggcagatctccaacgagctctacatcacggtgagtacggtggagcagcacctcacccgggtgtaccggaagctggacgtcaagagccgcacggacctgccgaaccggctcatggcccttgccgagccgatggcctag

SEQ ID NO: 2 - protein, 958 aa, Streptomyces ambofaciens
MLVHRDEQLAHLRWAFHACEKHGHGQVALVTGAVGSGKTQVLETFGEWAATAGGQVLSAAGSRAEQGLHFGVLGQLLHSARLKPEGIREVEELIREVALAVPTAEARSGAAPETLAEALTADGLWAPLLRTAFDTFLTLAGRGPLVLAVDDLQHTDAASLHCLLYVTRRLRNARIMVLLSEATTLRPAHPLFHAELRSLPRFTRVTLPLLTADSVSRLLGEEAGTPDVRGDGAGAPDVREEAGRILRVTGGNPLLSQALVDERAHRGTDSAPDTGARAGDTFERAVLDCLYRHEPGVRLVAQALAALDRPASPELLGQLLDVLPDSTVPAVRVLEGAGLVHAGRLRHPRIVLAVRSDMPAEERRRLHQRAAEVLHENGAEASVVAEHLVASAWTDGAWVVPVLRDAAAHALSTGRPDHAAACLRLGGRAETDKDRRNSLMAMLITARWQVNPLAVTGQVSQLVEAARADESSPSTAVSAVPYLLWQGRAEEAAEAISGCGAGDHGPASNELRLTRLLIALSHPDYLGTVREDPSSWTRAATAPDSASPLLQAVSVLGNALMPTGGVDTVAAAEQLLERHHTDSGSLGLLTAPLLALLCAGPADRVAVWGERLLARQGVQHTPAWRGVIRAIHAEATLRLGDMDAAERGARLALEDLPAPAWGVAVGGPLGTLITCTTESGRLFEAERWLSQPVPAGVFRTPVGAHYLIARGRHHLAMGQHQAAAADLHRSGELVRSWGIDVAGLVPWRLELARVQLSLGHRTHAAQLLQEQLQVSRGLDDRTRGRALRLLASTATPVLRRKLLSKAVTVLQACSDQQELARALSDTSQTLPQTGDAPRARLFVRRAGRLAQASGRAPALPQQVQRRSAPSDAAPTGGSSSDASSDGFESRDQDGLLSEAERRVAVLAARGRTNRQISNELYITVSTVEQHLTRVYRKLDVKSRTDLPNRLMALAEPMA

SEQ ID NO: 3 - DNA, 1131 bp, Streptomyces ambofaciens
atgctggtccatcgagacgaacaactggcccatctacgctgggcgttccacgcctgtgagaagcacggacacggacaggtggcgctggtcaccggcgctgtgggcagcggtaagacccaggtgctggagaccttcggggagtgggctgccaccgccggtgggcaggtgctgagcgccgccgggtcacgcgccgagcagggactccatttcggagtgctcgggcaactcctgcacagcgcgcggctcaaaccggaggggatccgggaggtcgaggagctgatacgcgaggtcgcgctggcggtgcccacggcggaggcccgaagcggcgcggcgcccgagaccttagccgaggccctgacagcggacgggctctgggcgccactgctgcggaccgccttcgacacgttcctgaccctggccggccgagggcctctggtgctggcggtcgacgacctccagcacacggacgccgcctcactgcactgtctgctctacgtcacccgaagactcagaaacgcacgcatcatggtgctgctctcggaggcgacgacgctgcgtccggcgcatccgctcttccacgcggagctgcgcagcctgcctcgcttcacccgggtcaccctgccgctcctcaccgccgactccgtctcccggctgctcggcgaggaggccgggacacccgacgtacgcggcgacggggccggggcaccggacgtacgcgaggaggccgggcggatcctgagggtgaccggcggcaatccgctgctgtcccaggcgctggtcgacgaacgggcccaccgcgggacggacagcgcccccgataccggagcgagggccggcgacaccttcgagagggccgtgctcgactgcctctatcgccacgagccgggcgttcgcctggtggcccaggcgctcgccgcgctggaccgcccggcctcaccggagctgctcggtcaactgctcgacgtgctgcccgactccaccgtgcccgccgtccgcgtcctcgagggcgcggggctcgtccacgcgggccggctgcgccatccacggatcgtgctcgcggtgcgttccgacatgccggccgaggaacggcggcggctgcaccagcgggcggccgaggtgttgcacgagaac

SEQ ID NO: 4 - protein, 377 aa, Streptomyces ambofaciens
MLVHRDEQLAHLRWAFHACEKHGHGQVALVTGAVGSGKTQVLETFGEWAATAGGQVLSAAGSRAEQGLHFGVLGQLLHSARLKPEGIREVEELIREVALAVPTAEARSGAAPETLAEALTADGLWAPLLRTAFDTFLTLAGRGPLVLAVDDLQHTDAASLHCLLYVTRRLRNARIMVLLSEATTLRPAHPLFHAELRSLPRFTRVTLPLLTADSVSRLLGEEAGTPDVRGDGAGAPDVREEAGRILRVTGGNPLLSQALVDERAHRGTDSAPDTGARAGDTFERAVLDCLYRHEPGVRLVAQALAALDRPASPELLGQLLDVLPDSTVPAVRVLEGAGLVHAGRLRHPRIVLAVRSDMPAEERRRLHQRAAEVLHEN

SEQ ID NO: 5 - DNA, 153 bp, Streptomyces ambofaciens
ctgagcgaggccgagcgaagggtcgcggtactggcggcacgggggcgtaccaaccggcagatctccaacgagctctacatcacggtgagtacggtggagcagcacctcacccgggtgtaccggaagctggacgtcaagagccgcacggacctg

SEQ ID NO: 6 - protein, 51 aa, Streptomyces ambofaciens
LSEAERRVAVLAARGRTNRQISNELYITVSTVEQHLTRVYRKLDVKSRTDL